(19) United States
(12) Patent Application Publication
(10) Pub. No.: US 2007/0106177 A1
(43) Pub. Date: May 10, 2007

(54) Apparatus for collecting and calculating quantity of patient fluid loss and method of using same

(76) Inventor: Yoshito Hama, Tokyo (JP)

Correspondence Address: FULWIDER PATTON LEE & UTECHT, LLP, 200 OCEANGATE, SUITE 1550, LONG BEACH, CA, 90802, US

(21) Appl. No.: 11/247,391

(22) Filed: Oct. 11, 2005

(51) Int. Cl. A61B 5/00; A61M 1/00

(52) U.S. Cl. 600/573; 604/318; 604/322

(57) ABSTRACT

A monitoring system including a control unit having a weight scale for carrying a collection bag configured with a bottom wall distended in a rounded concave shape, the central unit being responsive to the amount of fluid collected to produce a read-out which may be recorded or observed by a technician. In one embodiment, the system includes a hook suspended from the weight scale and projecting downwardly through a slot in a bottom wall to hook to the container.

### FIELD OF THE INVENTION

The present invention relates to a method and apparatus for measuring the quantity of fluids collected from a patient such as urine, saliva and/or blood and to thereby collect clinical information on the condition of the patient.

### BACKGROUND OF THE INVENTION

### Description of the Prior Art

In the treatment of patients recovering from an illness or who have been involved in an injury or, undergone surgery or may otherwise be involved in a critical condition it is understood that the volume of body fluid (e.g. urine, blood, etc.) loss is an important clinical indicator in assessing the patients condition and selecting the protocol for treating the patient. The quantity of such fluid that is of interest in detecting any imbalance which may be indicative of organ perfusion, or other abnormalities of the patient. It has been common practice to collect such body fluid in a container, such as a flexible wall bag made of a half-transparent material on the front thereof having graduations intended to correspond with the volume of liquid that may have been collected in the container.

A registered nurse is responsible for periodically reading the meter and recording his or her readings to make a record of the quantity of fluid in the container. During an operation, a nurse or medical technician typically monitor the amounts of liquids administered either on a continuous basis, as during surgery or at short intervals of, for instance 5 minute periods for patents subject to less critical care. To control the metabolism of intensive care patients, it is a nurses responsibility to, at regular intervals, monitor the liquid outputs from a patient, and for less critical post-operative patients, and recovering patients, make the rounds in hospital or clinic to read the gauge on the individual containers and hand record the information in the medical record of the various patients. This approach suffers the shortcoming that the reading of the information from the scale or meter on the container is highly subjective leaving the nurse with a certain degree of independent judgment as to how precisely the scale should be read. The reliability of the readings is further drawn into questions by the fact that the bag, whether suspended from a pole, adjacent an operating table or bed, may be disoriented and/or the walls thereof not uniformly and fully distended, thus allowing the nurse further independent judgment on whether to undertake the task of straightening the bag and seeking to settle the fluid at the bottom to allow for a more accurate reading.

The manual measurement of fluid volume is time-consuming, involving unclean procedures, and subjects the nurses' to technical and visual inaccuracies. The procedure is such that, in seeking to take the measurement, the conscientious nurse will avoid coming into direct contact with the collection bag to avoid direct exposure to any residual fluid contaminating the exterior. This problem is exacerbated by the fact that if, the collection bags are somewhat disoriented, maybe suspended at an angle from the vertical thus raising the risk of a false reading, the nurse will shift or jiggle the bag to settle the liquid toward the bottom in effort to render the visual gauge more accurate thereby incurring the risk of infection from any contaminated fluid.

Often times, during the night shift or when the illumination is otherwise lacking, it is also necessary to illuminate the gauge on the bag with a flashlight in effort to secure an accurate reading and shine the light on the record. This task can further distract attention from the task and adds to the challenge of seeking to manipulate the bag into vertical orientation and settling of the liquid thereby adding to the risk of error and/or physical contact with any contaminated fluid that may have spilled or leaked from the bag.

It will be appreciated that, with the threat of infections, care must be exercised in dealing with the disposal of bio hazardous bags and in complying with the associated health laws, all without the time constraints placed on hospital staff limiting the time allotted to such tasks.

With present day concerns over nursing staff shortage in the healthcare industry, nursing can be overtaxed thus raising a need for a reliable system for the fully automatic measurement of excreted liquid volume without requiring a reading of the measurements from a scale on the bag and without actually coming into contact with the surface of the collection bag. It is recognized that the task is often complicated by the fact the schedule of a busy nursing department may be such that it allows for emptying of the bag only when full or nearly overflowing often with a load or 2 to 3 kg thus, when coupled with the often overburdened schedule of nurses, inviting error and contact with fluid from a possibly infected patient.

Efforts have been made to provide meters to monitor fluid flow and for automatically detecting fluid collections. In this regard, it has been proposed to automatically measure the output of urine. Such a device is reported 25th International Symposium on Intensive Care and Emergency Medicine, Brussels, Belgium, 21-25, March 2005 as a comparative study on an electronic urine meter offered promoted under the trademark UREXACT. Such a meter is believed to rely on flow rate and fails to take into account the absolute weight of the fluid collected, a measurement that is most accurately reflective of the mass and volume of the output fluid.

C.R. Bard Company has proposed a system under the trade designation CRITICORE System for measuring urine output and to store data relating to measurements taken at time intervals and to store those measurements and the accumulated collection of output. It has been electronic urinometers (UROTRACK PLUS 220) with an RS-232 output port to rapidly record output data. It is believed this systems relies on flow rates and fails to take into account the absolute weight of the fluid collected and to utilize highly stable collection bag suspension fixtures.

In some of my own previous work, I proposed sensing the weight of fluid in a conventional rectangular-in-vertical cross section bag suspended from a weight cell carried on a horizontal rail as disclosed in my Japanese Patent No. 092049. While having certain advantages, I discovered that the system itself failed to provide prompt, consistent, reliable, and accurate readings. This conventional bag, particularly when loaded with a full load of fluid, would sometimes fail to load uniformly and could become hung in a lopsided manner thus detracting from the accuracy of the load cell reading. I have also discovered that highly sensitive weight monitoring systems can be negatively impacted by any instability in the suspension of the container from the load cell, as by suspension from an unstabilized hook or over the edge of a support table.

Thus, there exists a need for improvement in fluid monitoring systems and to balance a collection bag to uniformly collect fluid for reliable weighing thereof and to read the weight of the fluid collected without the need to manipulate or take readings from the bag itself.

This need, in its various aspects, is for a fully automatic fluid measuring system to take the fluid readings (1) without the necessity of contacting the bag or visually reading the measurements from the bag (2) with a central processing unit housed in a housing for a weight cell to suspend a bag in a stable manner and, in some embodiments, with (3) a digital display panel to indicate the measured data in real time or in a time-series manner as needed, which can be displayed either in the actual weight data or quantitative data converted from the weight and (4) a bag suspension device for collecting the fluid centrally directly under a suspension point and for, as the level of the fluid rises, spreading the fluid uniformly outwardly in the horizontal directions on opposite sides of a vertical centerline.

### SUMMARY OF THE INVENTION

In one aspect, the system of the present invention includes a housing mounting load cell weight meter. The weight meter includes a suspension hook depending from the bottom to project through a slot in a support shelf to hang the collection bag in stabilized relationship relative to the load cell.

One aspect of the present invention is characterized by a system for automatically weighing fluid collected in a collection container formed with horizontally extending front and rear vertical walls, symmetrical about a vertical centerline, and cooperating to form a rounded bottom wall and arranged to be supported along such centerline and a load cell for sensing the weight thereof and including a read-out indicative of the volume of fluid collected.

In another aspect, the present invention is characterized by a support table formed with a through slot for suspension of a hook or hanger carried from the load cell and suspending the container to gradually collect fluid supported in a stabilized manner for accurate electronic sensing of the mass of fluid being collected.

Other features and advantages of the invention will become apparent from the following detailed description, taken in conjunction with the accompanying drawings which illustrate, by way of example, the features of the invention.

### BRIEF DESCRIPTION OF THE DRAWINGS

FIG. 1 is a front view of a fluid collection container and central unit included in the system of the present invention;

FIG. 2 is a right side view of the collection bag and control unit shown in FIG. 1;

FIG. 2A is vertical sectional view, in an enlarged scale, taken along the lines 2A-2A of FIG. 1;

FIG. 3 is a front view, in enlarged scale of the bag shown in FIG. 2;

FIG. 4 is a side view, in enlarged scale, taken along the line 4-4 of FIG. 3;

FIG. 5 is a top view of the bag shown in FIG. 3;

FIG. 6 is a perspective view, in enlarged scale of a bag hanger included in the system shown if FIG. 1;

FIG. 7 is a partial front view, in a enlarged scale, of the bag and weight scale shown in FIG. 1;

FIG. 8 is a front view of a monitor which may be used with the system of the present invention;

FIG. 9 is a schematic view of a control unit incorporated in the system of the present invention for calculating fluid accumulated in the bag shown in FIG. 1;

FIG. 10 is a top plan view, in enlarged scale, of a control panel shown in FIG. 1;

FIG. 11 is a perspective view of a support shelf that may be utilized with the system shown in FIG. 1;

FIG. 12 is a perspective view similar to FIG. 11 but showing a control unit as depicted in FIG. 1;

FIG. 13 is a transverse sectional view taken along the line 13-13 of FIG. 12; and

FIG. 14 is schematic view of a central collection system that may be used with the system of the present invention.

### DETAILED DESCRIPTION OF THE INVENTION

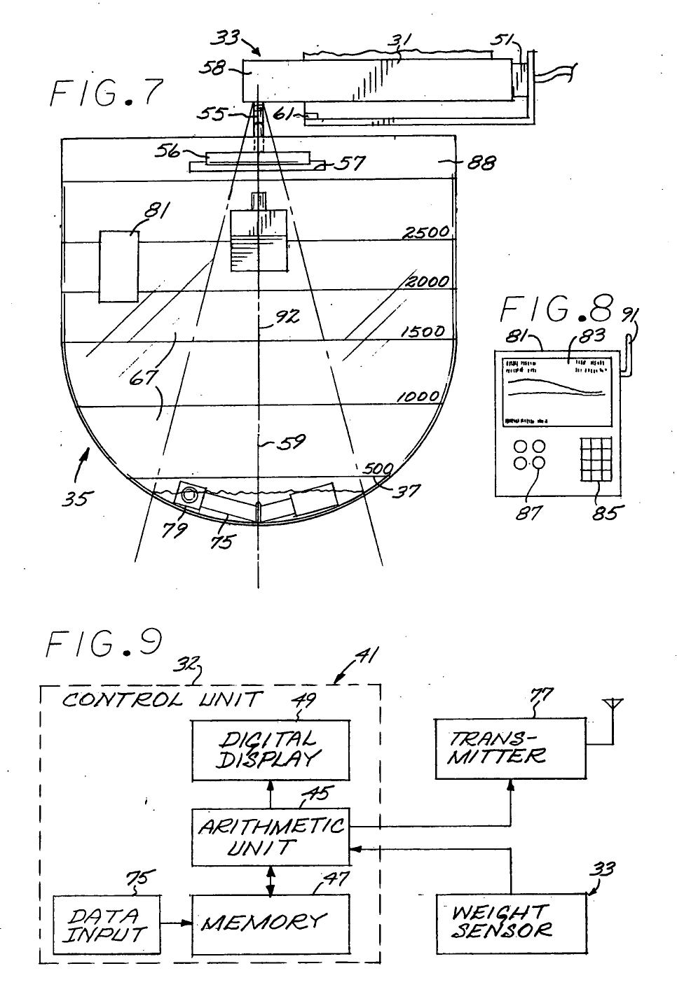

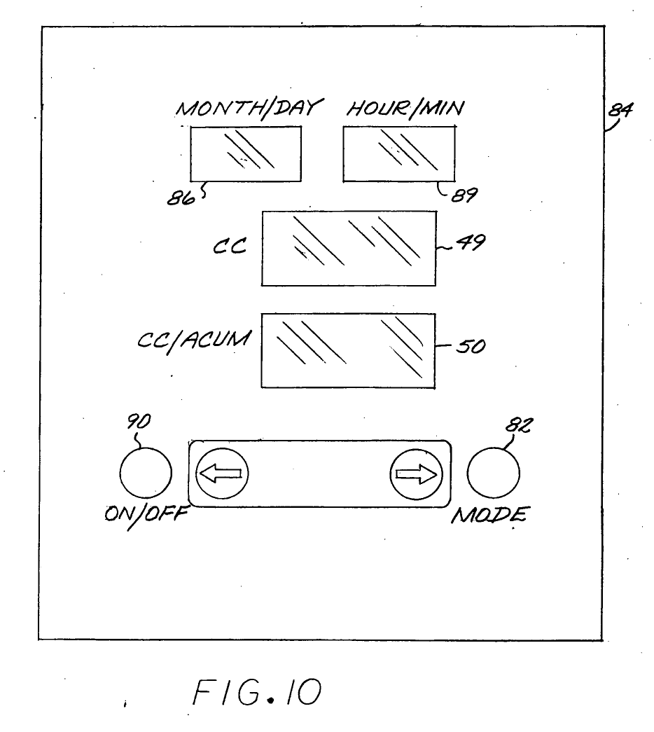

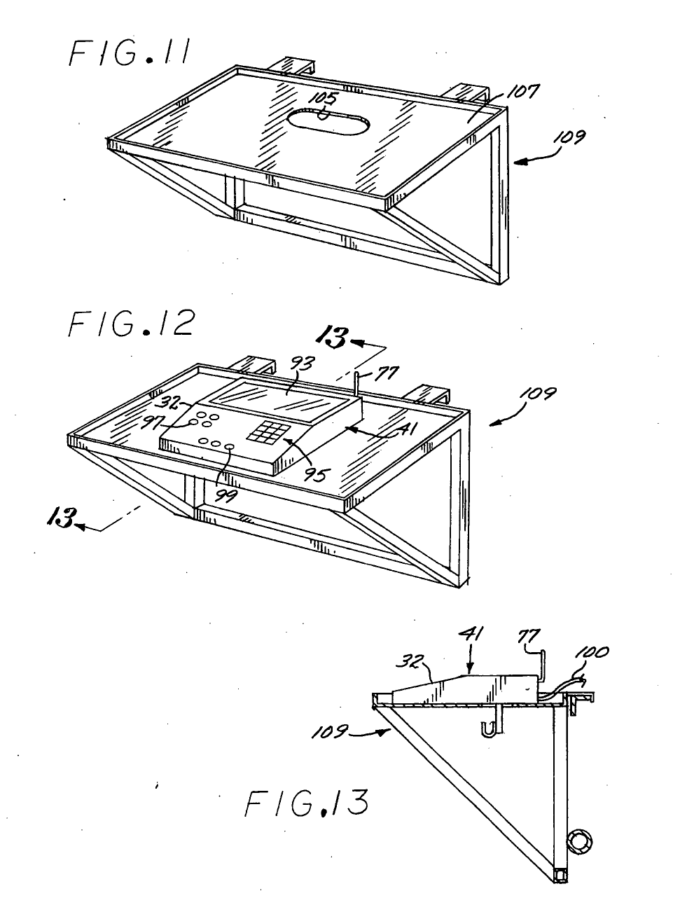

Referring to FIGS. 1 and 7, the body fluid collection and monitoring system of the present invention includes, generally, a control unit 41 connected with a load cell weight meter 33 in the form of a cantilever flexible beam 51 housed in a housing 31 and suspending from the free end thereof a fluid collection bag 35 having a laterally distended rounded bottom wall 37 configured symmetrical about a vertical centerline 59. Referring to FIG. 9, the control unit includes a converter 45 responsive to a signal from the weight sensor 33 and data from a memory 47 to generate an output signal which may be communicated to a display panel 49 (FIG. 12) to display an output corresponding with the weight calculated as being representative of the volume of fluid collected in the container bag 35.

The housing 31 may take many different forms and may, for instance, be in the form of an open frame to suspend a hanger 55. It is necessary that the weight scale provide quick response and accurate results. In the preferred embodiment, it includes a cantilever flexible beam 51 (FIG. 7) carrying hanger 55 mounting on its bottom end an upwardly opening horizontally elongated channel hook 56 for receipt in a through horizontal slot 57 centered along the vertical centerline 59 of the container 35.

In this regard the weight the cell 33 itself is selected to detect the deflection of the free end of such beam 51 to generate an electrical signal corresponding to the degree of deflection to instantaneously indicate the weight of fluid in such container. In my preferred embodiment, the weight cell is selected to provide an accuracy of within 3 grams and preferably within 1 gram. As will be appreciated by those skilled in the art, this scale can take many different forms, it only being important that it be highly accurate to produce reliable readings for electrical monitoring and processing by the system of the present invention. One eight cell that has proven effective is sold by A&D Company, LTD, Model No. ES-12ki and having an accuracy of within 1 gram and a weight capacity of 3,000 grams.

Mounted beneath the free end of the beam 51 is a pressure sensitive sensor 61 (FIG. 7) responsive to contact by the free end of such beam to communicate a signal to the central control unit 41 to alert the operator that the bag is full and should be replaced.

The collection bag 35 has a flexible transparent front wall defining a conventional fluid meter having vertically spaced graduations 67 there along to roughly correspond with the volume of fluid collected in the bottom of the container.

As will be appreciated, of course, the accuracy of the volume read from the graduations 67 will depend on the degree to which the bag is perfectly erect and the degree to which the front and back walls are spaced apart thus being unacceptable for reliable accurate readings. In this regard, because of the sensitivity of the load cell and the electrical controls, the accuracy of the weight or volume measured or calculated will be greatly effected by any movement of the bag or sloshing about of the liquid contained therein. Since the nurse responsible for monitoring the weighed measurements will soon become accustomed to the display of the measurements being extremely accurate, he or she will in the interest of time, tend to read the display relatively quickly thus inviting error from any non-steady state conditions, such as swinging or oscillation of the bag which tends to introduce erratic sensing and thus an inaccurate display.

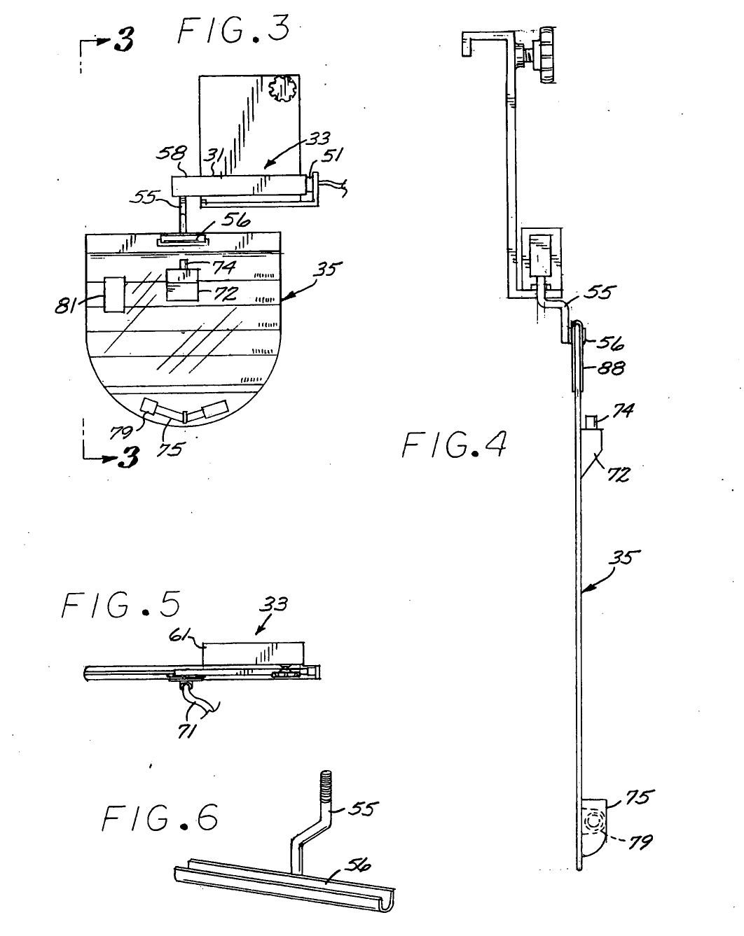

Referring to FIG. 4, in a preferred embodiment, the bag includes a rigid fluid inlet block 72 having a horizontal and vertical extent and configured with a vertical through passage which extends down from an upstanding central nipple 74 and angles inwardly to pass through the wall to the interior of the bag for flowing fluid under the weight of gravity from an inlet tube 71 connected to such nipple. This serves to create a fluid head generating a pressure between the front and rear walls of the bag tending to separate them and create a volume for the fluid. An outlet fitting 75 is mounted exteriorly at the bottom center of the front wall of the bag to be connected with a discharge tube 79.

Preferably, the individual bag includes an indicator bearing personalized data such as name, age, social security number and the like for the patient, as well as, an indication of the routing in the processing unit through which the information collected should be communicated. This indicator may be in the form of a bar code 81 (FIG. 1) or may be in the form of an embedded radio frequency bearing the identification and date, an integrated circuit tag to transmit a radio frequency or an engineered plastic film incorporated in the body of the bag itself. This information data may be transmitted through a reader directly to the control unit 41 to be recorded in a memory, compared with data recorded in the memory displayed or printed out on a graph. The identification data may carry intelligence to identify the routing for which the data is expected to be routed through the system or relating to the history of the bag itself and its intended disposal in a biologically safe manner. The data read may be compared with the desired routing previously programmed in the system and data collected for the purposes of control and security, as well as the inventory and disposition of the bag for safety purposes or even for defense legal purposes in the event of a claim or an inspection investigating the appropriate disposal of biological medical items and the record keeping related thereto for products deemed biologically hazardous.

The control unit may be formed by a printed or integrated circuit or may be driven by a software program and may be hardwired or may be wireless for receiving and transmitting data. The unit itself includes a converter 45 (FIG. 9) which performs the arithmetic on the weight signal from the weight cell 33 and is operative in response to input signals from the memory 47 to cooperate with the weight signal to generate a volume signal to be transmitted to the display 49 (FIG. 12). A data input device 75 may be preprogrammed and may have manual control buttons or knobs such that various information relating to different parameters such as the expected specific gravity of the fluid(s) being collected has urine, blood, excretion(s) and the dead weight of the bag container 35 and hanger 55 so that the output to the digital display will be modified to discount the dead weight and to reflect the accurate net weight and thus accurate volume of the fluid being measured.

In the preferred embodiment, the control unit includes a transmitter 77 (FIGS. 9 and 12) for transmitting a radio frequency signal corresponding with the output signal from the converter 45 for transmission to a remote receiver for the purposes of monitoring and or recording as will be described hereinafter.

Referring to FIGS. 1 and 12, the control unit may be housed in a frame defined by the portable control case 32, (FIG. 8) and incorporates a display panel 93, input buttons 95, control knobs 97 and indicator lights 99 associated with, for instance, the control indicator pressure switch 61 (FIG. 5).

Thus, the portable control unit so housed may be moved to different rooms or locations were the patient(s) are located and the various data input with the display panel 93 readily available for viewing the visual display results.

Referring to FIG. 1, the control unit 41 may be received slidably under an open front frame of a monitor shroud, generally designated 82, having a top wall in the form of a control panel 84 (FIG. 10) formed with a pair of windows 86 and 89 in the upper portion thereof for displaying the month and day and hour and minutes respectively (FIG. 10).

The control panel 84 also includes an on/off switch 90 (FIG. 10) and a mode switch 92 which may be rotary for selecting the mode in which the various outputs are to be displayed. As for instance, the display and window 49 may be in the form of weight, volume, graph or chart reflecting the history of the patient and the window 50 may display similar information for historical and analysis and record keeping purposes. The monitor 82 further mounts an antenna 94 for communicating radio frequency to or from a central network.

A remote handheld monitor device, generally designated 81 (FIG. 8), may also house all or a portion of the control unit 41 and incorporates a display panel 84 from which text, graphics and graphs may display personal information on the patient, text relating to the read-out and graphs showing the cumulative amount of fluid expelled and/or historical variations of quantities of fluid expelled. The monitor 81 may include input buttons 85 and knobs 87 to facilitate input of such data as would correspond with the input data 75 shown schematically in FIG. 9. Thus, the remote monitor 81 includes an antenna 91 for receiving the radio frequency signal from the transmitter 77 or transmitted to a central station.

Referring to FIGS. 1, 2A and 7, the bag 35, in the preferred embodiment, includes the transparent front wall and opaque or transparent back wall and is formed to be distended horizontally. Preferably, it is of generally a flexible construction but has mounted at the top end of a thin rigid or semi rigid horizontal stiffener strip 88 formed centrally with a horizontal through slot 57 for receipt of the hanger 56 (FIG. 7). The front and back walls join at the bottom to form a rounded bottom wall 35 which may be in the form of a semicircle as viewed from the front in FIG. 7 with the center 92 of its radius of curvature disposed on the vertical centerline 59 such that the symmetrically configured bag is balanced under the hanger 55 to be, in its suspended position, centered. As will be appreciated by those skilled in the art such bag may take many different forms, it only being important that the bottom wall be distended laterally and be configured to, when filled, maintain such bottom wall in its rounded configuration to thus cause the fluid collected at the bottom to form somewhat of a crescent shape flowing out horizontally on opposite sides of the centerline. The term rounded is intended to include any symmetrical rounded shape, such a circular, elliptical or parabolic, it only being important that it be rounded and symmetrical to, upon initial filling, collect the fluid centrally at the bottom but to, in the event of any bag movement which might momentarily disrupt fluid equilibrium raising the level up at one side of the bag, provided for flow uniformly back down the curved bottom wall toward the center of the bag to minimize any disruption.

As will be appreciated, this horizontally distended configuration of the bottom wall is maintained by the fact the flexible walls of the bag are suspended directly below from the horizontal stiffener bar 88 but, in other embodiments the bag may take on a configuration where the front or back wall or both themselves have sufficient structural stiffness in at least their upper portions to maintain the walls laterally distended under the load of fluid being loaded thereinto.

As fluid is received in the bag 35 it will collect and puddle along the semicircular bottom wall 37 (FIG. 7) tending to settle uniformly on the horizontally opposite sides of the centerline 59 and, as the volume grows, the top surface thereof will move upwardly and spread uniformly laterally outwardly to provide substantially equal masses or opposite sides of the centerline 59. It is important that the mass of fluid be spread gradually outwardly toward the lateral outer extent of the opposite edges of the bag as the level of fluid rises. As will be appreciated by those skilled in the art, the semi-cylindrically shaped bottom wall tends to collect the initial quantity of fluid near the centerline so as to weigh the bag down along such centerline tending to maintain the bag centered. As the bag fills, the rounded bottom wall will tend to maintain a major quantity of such fluid centered while the stiffening bar cooperates in supporting the walls of such bag from collapse toward the center thereby causing the fluid, as the level raises, to spread laterally outwardly toward the opposite lateral edges of the bag to thus distribute weight laterally outwardly to provide a relatively larger portion of the mass displaced laterally outwardly from such centerline as dictated by the weight of the fluid collected and the volume thereof. In practice, the bag is about 8 inches wide and 10 inches deep such that a fairly substantial volume of fluid may be collected.

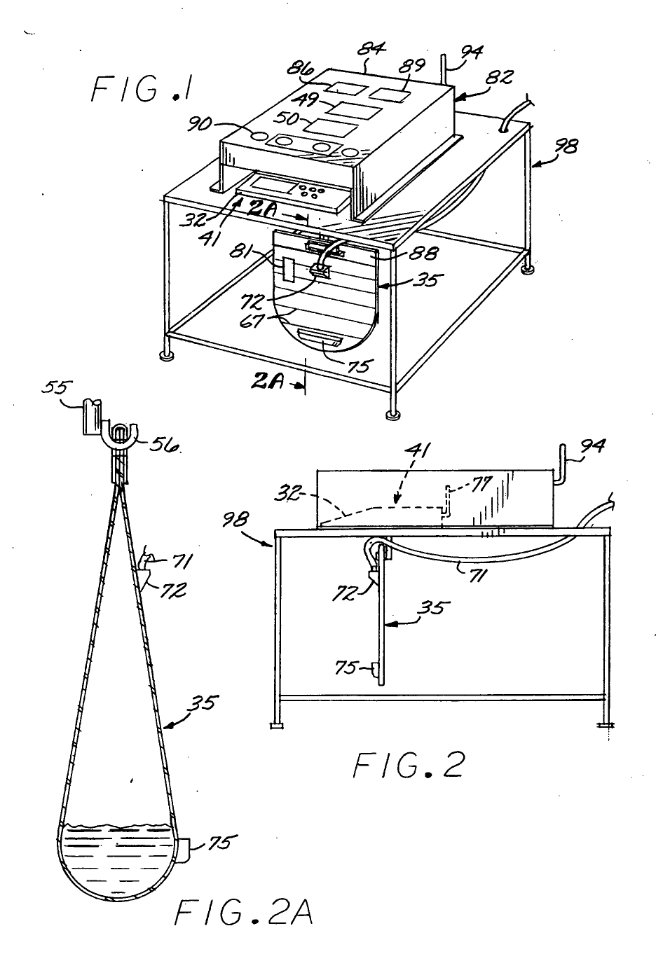

With reference to FIGS. 1 and 2, in use, the case 31 may be mounted on a support table, generally designated 98 which may have a slot formed in the top wall thereof for extension of the hanger 55 for suspending the bag 35.

The weight cell 51 includes an electrical sensor which communicates an electrical signal to a converter acting as the arithmetic unit 45 (FIG. 9). The memory 47 also communicates a signal corresponding with the particular specific gravity of fluid identified by the input and the circuitry in such converter and is operative to produce an electrical volume signal to the digital display 49 which is indicative of the volume of fluid collected in the container 35 for viewing by a nurse. As will be appreciated by those skilled in the art this read-out may take many different forms such as metric or English, volume, or weight or any other perceptible measurement corresponding with the weight of the fluid communicated. The output from the converter is also communicated to the transmitter 77 and the corresponding radio RF signal communicated to the antenna 91 of the monitor 81 so that the read-out may be viewed on the display panel 83 in text or graph form. The read-out will typically be recorded in the memory of the control unit 41 and/or in the monitor 81 so the history of the patient can be easily monitored by the technician or medical doctor having responsibility for the patient. This history may be displayed in the window 93 (FIG. 12) representing, for instance, the flow rate, accumulated volume or weight or, if desirable a data chart or graph representing the historical collection of fluid over selected periods of time such as the past several hours, days or even weeks.

The free end of the inlet tube 71 will be connected with one or more fluid collection devices, such as a Y-connector leading from a urine catheter or the like for collecting fluid from the patient to be introduced to the container bag 35. Referring to FIG. 8, data can be inputted to the control unit through the remote data input button 85 and knobs 87 or through controls on the unit 41 (FIG. 12) to select a preprogrammed specific gravity or gravities for the fluid(s) to be collected and a weight characteristic of the particular volume of bag container 35 selected so as to program that information into the converter 45.

In operation, it would be appreciated that a number of individual container bags 35 may be stored in inventory and when bags are withdrawn from inventory, a code may be entered on the bar code 81 (FIG. 7) corresponding with the data relating to the particular patient for which the bag is to be used. Furthermore, in some instances, the data in the bar code 81 will be operative to indicate a particular routing for data to be compiled for that particular patient, which routing may be compared with a routing intelligence stored in the CPU as a security check to be assured that the data is transmitted to the appropriate display, recorder or for additional processing. This data will be picked up by a bar code reader.

The CPU in the portable case 32 may be moved into position adjacent a particular patient to be treated, either supported from an operating table, from a custom table as at 98, from a custom shelf as at 109, or supported in any other desirable fashion, it being important that the support be relatively stable and that the container bag 35 be freely suspended such that the weight thereof will be supported from the hook 55. In this regard, when a bag is selected, the stiffener strip 88 may be hooked over the hook channel 56 by fitting it over the vertical flange of such channel (FIG. 6) and nesting the upper edge of the slot 57 (FIG. 7) into the bottom of the channel and centering the bag such that its own centerline 59 is directly under the hook. In some embodiments, the slot 57 is of a length corresponding with the length of the hook channel 56 so that when hooked in position, the bag itself will be centered. The inlet tube 71 will then be connected on one end with the nipple 74 projecting upwardly from the inlet block 72 and the opposite end connected with, for instance, a catheter or the like collecting liquid from the patient. In those embodiments including a monitor 82, the electrical lead 42 (FIG. 12) will be connected, the monitor activated, and the desired data input.

The on switch may then be depressed to actuate the CPU and any reference data inputted at 75 (FIG. 9) to be stored in memory, such as, for instance, personal information about the patient, specific gravity of the fluid expected to be collected and the like. As the fluid flows through the inlet tube 71 under the influence of gravity to the rigid inlet block 72 (FIG. 2A), it will flow downwardly and inwardly along the downwardly and inwardly inclined passage in such block to build up a modest hydraulic head seeking to enter the bag to press against the inner surface of the front wall of the bag so that the gravitational weight thereof will tend to press the front wall of such bag away from the back wall to thus overcome any surface tension or stiffness in the walls tending to hold the two walls together. This will then allow the liquid to flow unrestricted downwardly within the interior of the bag to thus puddle against the semicircular bottom wall as shown in FIG. 2A. The weight cell 33 will be operative to, in response to deflection of the cantilever deflection, produce a proportional electrical signal to be communicated to the converter 45 (FIG. 9) which will cooperate with the memory 47 to generate a electrical signal corresponding with any preprogrammed information, such as the weight of the empty bag 35, specific gravity of the fluid or combination of fluids to thus generate a display signal to be communicated to the digital display 49 for display of, for instance, the total weight of fluid collected or the volume thereof. The CPU 41 will be further operative to, in response to the particular mode selected, generate a signal indicative of the rate at which the fluid is being collected or, for instance, a tally signal representative of the total accumulated fluid collected over a selected such as 1 to 3 or 8 hours, previous day or so or even the past week to be displayed in the display window 49 (FIG. 12). It will be appreciated as the fluid is collected in the container bag 35, the general volume thereof will be roughly indicated by the graduation 67 on the transparent face of the bag so the nurse will have a general indication of the current volume to allow him or her to very roughly estimate the volume collected.

In the preferred embodiment, the memory 47 is operative to continuously record the pertinent data, such as date and time, progressive accumulation of fluid, variations in the rate of accumulation and data relating to the identity of the patient and, in some instances the patient's injury.

It will be appreciated that, as the volume of fluid grows in the bag 35, the horizontal stiffener strip 88 at the top of the bag (FIG. 5) will be operative to maintain the laterally extending walls of the bag suspended in a uniform manner across the entire width in the plane of the paper as viewed in FIG. 7 so as to prevent unwanted sagging of the walls of the bag to thus provide support against the mass of fluid inherently seeking to form a spherical ball at the bottom of the flexible wall bag without horizontal distribution thereof and tending to, with any disturbance, swing in pendulum fashion thereby detracting from a static condition which would lead to unwanted variations in the weight served by the weight cell. The stiffener strip 88 will thus maintain the front and back, bag walls distended laterally outwardly in sling fashion to thus maintain the bottom wall, when viewed in the plane of the paper in FIG. 7, in a generally semicircular configuration to direct the initial flow of fluid to the center of the bottom wall to maintain relatively even distribution of the fluid on opposite sides of the longitudinal centerline as the level of the fluid raises. Consequently, the fluid will tend to collect and the upper level thereof flow outwardly and upwardly in the bag along the opposite edges thus accumulate in a volume which is relatively thin in a direction perpendicular to the paper as viewed in FIG. 7 but having a lateral extent in the direction of the longitudinal axis of the stiffener which extends fully to the vertical planes of the respective ends of such stiffener.

The resultant volume will thus produce a mass which is elongated horizontally in the vertical plane of such stiffener. Thus, as the mass of fluid grows in the bag, the masses on the opposite sides of the centerline will become greater. These masses then present their own moments of inertia resisting oscillation in the plane of the paper as viewed in FIG. 7. These moments of inertia will have relatively large horizontal moment arms from the juncture of the centerline with the hanger to the theoretical respective centers of gravity for the opposite halves of the masses of fluid to afford relatively large inertial moments to cooperate with the stiffener strip 88 and the channel hook 57 (FIG. 7) to resist oscillation in the plane of the paper in FIG. 7 tending to maintain stability and minimize erratic readings.

If one were to analyze the static forces of this mass as the bag fills, it is useful to consider the respective masses on the opposite sides of the vertical centerline 59. First, as will be understood by those skilled in the art, with the initial collection of fluid at the bottom of the centerline, any disturbance of the bag or sloshing of the fluid will have little effect on the bag as, with the weight of fluid generally funneled to the center bottom, any such disturbance will be quickly dampened out. As the level of the fluid raises, the upper levels thereof will tend to spread out laterally in the plane of the paper under the stabilizer bar. It will be appreciated that, while the mass is becoming greater, the theoretical centers of gravity of the two halves of the total mass in the bag will be located in respective vertical planes located a distance equal to one quarter the total width of the bag from such centerline. The distances from those respective theoretical centers of gravity to the horizontal center of the hanger thus represents the theoretical moment arms for the inertia afforded by each half of the total mass. The moments of inertia of the respective halves of the total such mass thus tend to resist rotation of such bag about the horizontal center of such channel hook with a force proportional to the distance such theoretical centers of inertia are spaced from the theoretical center of rotation for the bag (i.e., horizontal center of the stabilizer bar 88).

As the bag gradually fills with fluid, the CPU is operative to maintain an accurate record thereof. When the fluid approaches or exceeds the level of the inlet block 72 (FIG. 7) the cumulative weight thereof will be sufficient to flex the weight scale lever arm to the point where it contacts the pressure switch 61 to thus generate an audible alarm and optical signal at the lamp 99 to alert the operator that the bag should be emptied. The bag may conveniently be emptied by opening the outlet tube 79 thus allowing the bag to drain into a disposal receptacle or the like, all without any need for the nurse to touch the or even come into close proximity with the bag.

As will be understood by those skilled in the art, the data from the bar codes 81 may be transmitted to memory for a permanent record so that the whereabouts of any particular bag may be tracked or, compared with prestored information to produce an inventory read-out alerting the operator of the total number of bags used from inventory and the number remaining. In some instances, the system is programmed so that the operator must input data reflecting the time and location where a bag was disposed of.

It will be appreciated that, to promote the efficiency of the hospital or convalescent home staff, it is desirable that personnel might move around and perform additional duties while still having access to the information as to fluid collected from one or more patients. To this end, the portable hand held unit 81 may be carried in the technicians pocket and may be from time to time reviewed to monitor the signal transmitted from the antenna 94 to the antenna 91 (FIGS. 6 and 12) to thus maintain a constant update. The hand held unit 81 may be responsive to various frequencies such that different frequencies admitted from the antenna 77 of different monitors for the various different patient's may be individually monitored.

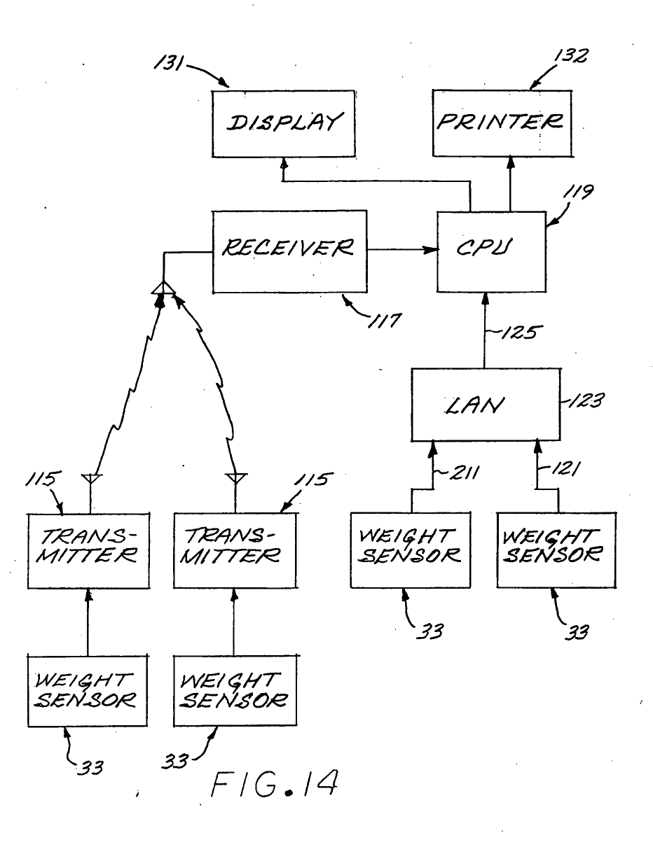

In some instances, the system of the present invention will tap into the preexisting network 123 (FIG. 14) in the hospital facility and the weight sensors 33 associated with the various monitors monitoring various individual patient's transmit through the landline to the CPU 119 for collection of the data display, recording and if desirable the printing thereof through the printer 132. In other applications, the information from the sensors will be transmitted to the transmitters 115 to a receiver 117 to be fed into the CPU 119.

A fourth embodiment of the present invention is shown in FIG. 14 that includes multiple weight cells 33 for sensing fluid loss by multiple patients, each including a respective transmitter generally designated 115 for transmitting RF signals to a receiver 117 connected with a central processing unit, generally designated 119. Also, connected with the CPU 119 are another pair of weight cells 33 connected by hard lines 121 to a local area network 123 such as a previously installed network within a hospital or other medical facility to collect data and communicate through a line 125 to the CPU 119. The CPU 119 is operative to receive the individual signals from the various weight sensors, which signals will typically carry a identifying code associated with each of the patient's the input data from the respective indicators 81 to be processed along with the weight signals for communication to a display, generally designated 131, to be monitored in a central monitor area by a nurse or other technician. Concurrently, the collected data will be transmitted to a printer, generally designated 131 which will, either on demand or continuously, print out the data.

With the arrangement of FIG. 14 it will be appreciated a selected number of weight sensors may be distributed throughout the various hospital wards, ICU, ER and the like to weigh fluid from the individual patient's and communicate the resultant information to the CPU for monitoring by a single medical technician who might have a split screen monitor or possibly a sequence through the data from the various individual patient's. The monitor may be display the data relating to the individual patient's, there personal information and the date and various relevant dates such as date of surgery and date of the monitoring.

The system of the present invention has been used in a clinical setting and has proven to provide rapid and accurate responses allowing the nurse to quickly observe data on the volume of fluid collected and without physical contact with the bag.

As will be appreciated by those skilled in the art, the device of the present invention may take many different forms without departing from the spirit of the invention as defined in the amended claims. For instance, some medical facilities or departments may prefer more functional support table, such as roller mounted cabinets with the CPU incorporated in an upper compartment having a top wall in the form of a control panel, with the collection bag suspended internally behind closable doors. In other embodiments, device may be in the form of a portable table or counter mounted housing comparable to a coffee maker with an overhang mounting the weight scale to suspend a collection bag shrouded in an encircling, open front shroud.

From the foregoing, it will be appreciated that the system of the present invention provides a convenient inexpensive, safe, reliable, and accurate means for collecting fluid from individual or groups of patient's to automatically and instantaneously monitor fluid loss directly from a load cell or from a remote station, without necessity of tracking the collection bag during the reading step or even during disposal thus providing the accurate and safe monitoring and recording of the critical information on a continuous basis. The collection bag of the present invention provides a particularly effective and convenient implement to suspend from a weight scale to automatically collect fluid in a relatively stable manner without readings even in the most sensitive monitoring system.

I claim:

1. An automatic system for precise calculation of the volume of body fluid expelled from a patient comprising:
an upright collection container for collecting the fluid, elongated in one horizontal plane, symmetrical about a vertical centerline and formed with a downwardly concave, rounded bottom wall projecting in the one horizontal plane;
a frame;
a weight scale in the frame and responsive to the weight of fluid in the container to generate an electrical signal proportional thereto;
a hanger for hanging the container from the load cell;
a control unit, in communication with the weight scale for receiving the electrical signal and responsive thereto to generate a read-out and including a read-out display device to convert the read-out signal to a perceptable signal whereby the container may be hung from the hanger to be supported upright and distended in the one horizontal place such that fluid collected therein will initially collect about the centerline at the bottom of the concave bottom wall and, as collection continues, will spread out horizontally in the one horizontal plane with the control unit operative in response to the control signal to display a perceptible signal indicative of the mass of fluid collected.

2. The system of claim 1 wherein:
the control unit includes a memory and is operable to record the read-out signal.

3. The system of claim 1 wherein:
the control unit includes a memory programmed with data on the specific gravity of the fluid and the unit is responsive to the read-out signal and the data to display a sum indicative of the volume of the fluid collected in the container.

4. A system of claim 1 wherein:
the container is constructed with flexible front and back walls that curve downwardly and inwardly toward one another on their laterally opposite sides and are joined together to form the bottom wall in the configuration of a semi-circle.

5. A system of claim 1 wherein:
the control unit includes a comparator for receiving the display signal and operative at selected time intervals to store the magnitude of the display signal and is further responsive to, at predetermined time intervals, detect the magnitude of the display signal and compare it with the previously stored display signal.

6. A system of claim 1 wherein:
the control unit includes an accumulator responsive to the display signal to generate a tally signal; and
the display device includes a display responsive to the tally signal to display a signal representative of the cumulative amount of fluid collected in the container.

7. A system of claim 1 wherein:
the frame includes a sensor disposed adjacent the container and responsive to accumulation of a predetermined volume of the fluid therein to generate a full signal.

8. A system of claim 1 wherein:
the weight scale includes a flex beam constructed to flex in proportion to the weight of the fluid in the container.

9. The system of claim 1 wherein:
the collection container includes an identifier for recording personalized data; and
the control unit includes a reader for reading the personalized data.

10. The system of claim 9 wherein:
the identifier is a bar code; and
the reader is a bar code reader.

11. The system of claim 9 wherein:
the control units include multiple routes for connection of the electrical signal; and
the identifier is configured to store data characteristic of selected ones of the routes for distribution of information.

12. The system of claim 9 wherein:
the identifier is in the form of a radio frequency identification device.

13. The system of claim 9 wherein:
the identifier in the form of an integrated circuit to transmit a signal to the reader.

14. The system of claim 1 wherein:
the frame includes a housing including a horizontal bottom wall configured with an opening; and
the hanger includes a hook connected with the load cell for projecting through the slot to hook to the container.

15. The system of claim 1 wherein:
the control unit includes a memory and a data input programmable with data corresponding with weight of the container when empty and the specific gravity of the fluid to be collected.

16. The system of claim 1 wherein:
the container includes a wall formed with vertically extending transparent meter window and measurement indices spaced vertically there along approximating the volume of fluid collected in the container.

17. The system of claim 1 wherein:
the control unit includes a memory and data input coupled with the memory and operable to input the specific gravity of selected body fluids to be collected in the container.

18. The system of claim 1 wherein:
the control unit includes a transmitter for transmitting a radio frequency signal proportional to the electrical signal; and
the system includes a central station with a receiver for receiving the radio frequency signal and responsive thereto to generate a display signal.

19. The system of claim 1 that includes:
a predetermined number of the containers for receiving body fluids;
a predetermined number of the weight scale disposed in weight transfer relationship with the respective containers and operative in response to the weight of fluids in the respective cells to generate respective electrical signals;
a predetermined number of transmitters responsive to the respective electrical signals for transmitting respective RF signals, including identification signals; and
the control unit including a receiver for receiving the RF signals and identification detectors for detecting the identification signals in the respective RF signals.

20. The system of claim 1 that includes:
a printer connected with the control unit and responsive to the read-out signal to print data corresponding with the read-out signal.

21. The system of claim 1 that includes:
a predetermined number of the containers;
a predetermined number of the weight scales responsive to the weight or fluid in the respective containers to generate respective electrical signals proportional thereto and wherein;
the system includes a control station in electrical connection with the respective weight scales for receiving the respective electrical signals and includes means responsive thereto for generating signals proportional thereto.

22. The system of claim 1 wherein:
the container is in a form of a flexible bag.

23. The system of claim 1 wherein:
the container includes an outlet formed in the bottom portion thereof.

24. The system of claim 1 wherein:
the container includes vertical flexible front and back walls, an inlet in the upper portion of one of the walls and an outlet in the lower portion of the one wall;
a rigid inlet block mounted over the inlet, projecting outwardly from the one wall, and including a nipple on the top extremity thereof and a passage extending downwardly in the block and angling through the one wall; and
an outlet in the lower portion of the container.

25. The system of claim 1 wherein:
the control unit includes a transmitter for receiving the electrical signal and for transmitting radio frequency corresponding thereto; and
the system includes a hand held monitor including an RF receiver for receiving the RF signal and including a display panel for displaying displayed data corresponding with the RF signal received.

26. The system of claim 1 wherein:
the control unit includes a mode switch for selecting a mode of display corresponding with the display signal.

27. The system of claim 1 wherein:
the container is in form of a flexible bag and is configured symmetrical about a vertical centerline and further includes a horizontally elongated stiffener bar in the upper portion thereof for suspending the walls of the bag therefrom.

28. The system of claim 27 wherein:
the stiffener bar includes a horizontally elongated slot; and
the hanger includes a suspension hook including a horizontally elongated upwardly opening channel for receipt in the slot and for nesting of the upper edge of the slot therein to cooperate in maintaining the stiffener bar in a horizontal plane.

29. A body fluid collection bag for suspension from a hook connected with a weight scale and comprising:
front and back walls connected along their edges to cooperate in forming a vertical flexible container distended in a horizontal plane and configured at its bottom extremity with a semicircular bottom wall disposed in the horizontal plane, the container being symmetrical about a vertical centerline;
a horizontal stiffener bar along the upper extremity of the bag for supporting the front and back walls and cooperating in maintaining the bottom wall distended in its semicircular configuration;
an attachment disposed centrally on the stiffener bar for connecting with the hook;
an inlet fitting in the upper portion of the bag; and
an outlet fitting in the lower portion of the bag.

30. A system for precise calculation of the volume of body fluid expelled from a patient and comprising;
a frame;
an upright flexible wall collection container formed with a front wall configured with a vertical translucent meter window having graduations spaced vertically there along corresponding for alignment with the top surface of the fluid in the container to approximately represent the volume of the fluid collected, the container formed with a semicircular bottom wall and including a discharge outlet in the lower portion of the front wall and further including an inlet in the upper portion thereof for introducing fluid from the patient;
an inlet block overlying the inlet and being formed with a vertical passage angled toward and joining the inlet;
a weight scale interposed between the frame and container and including a cantilever flexible beam having a hook on the free end thereof for suspending the container and responsive to the weight of fluid in the container to generate an electrical signal;
a central processing unit including a converter connected with the weight scale and including a memory having an electrical specify gravity signal and being responsive to the electrical signal and specify gravity signal to generate an electrical volume signal; and
a display device including a display panel and responsive to the volume signal to generate display signal on the display panel corresponding with the volume signal.

31. A method of measuring the fluid expelled from a patient's body including;
selecting a vertical fluid container formed to extend horizontally in one vertical plane and having an inlet tube in the upper portion, symmetrical about a vertical centerline, and including a concave rounded bottom wall;
selecting a weight scale;
suspending the container from the weight scale from a point along the centerline;
accumulating the fluid in the bag to gradually fill form the bottom wall at the centerline to flow horizontally outwardly and upwardly along the surface of the concave bottom wall rounded in the one vertical plane;
sensing the weight of the fluid by the weight scale;
electrically monitoring the weight of the fluid from the weight scale and utilizing the weight sensed to generate a display.

32. A system for precise measurement of excreted body fluids comprising:
a vertically extending container for collecting the fluid, extending horizontally and being symmetrical about a vertical centerline;
a frame for supporting the container and including an opening and bottom thereof;
a weight scale in the frame;
a hanger suspended from the weight scale and projecting downwardly through the opening;
a control unit in the frame and connected with the weight scale and being operative in response to the magnitude of weight applied to such scale to generate an electrical signal and being operative in response to such electrical signal to generate a display signal; and
a support table and including a platform for supporting the frame and being formed with a slot for depending there through of the hook to support the container from such hook freely suspended from the weight scale and projecting below the level of the platform.
